(19) United States
(12) Patent Application Publication
(10) Pub. No.: US 2026/0306821 A1
(43) Pub. Date: Oct. 8, 2026

(54) DRIPLESS FLUID COUPLING DEVICES AND METHODS OF USE

(71) Applicant: EMD Millipore Corporation, Burlington, MA (US)

(72) Inventors: James Ormond, Belmont, MA (US); Heejin Lee, Bedford, MA (US)

(21) Appl. No.: 19/484,231

(22) PCT Filed: Jul. 25, 2024
(86) PCT No.: PCT/US2024/039616

§ 371 Date: Nov. 13, 2025

Related U.S. Application Data
(60) Provisional application No. 63/578,815, filed on Aug. 25, 2023.

(51) Int. Cl. A61M 39/10; A61M 39/24; A61M 39/26

(52) U.S. Cl. CPC A61M 39/1011; A61M 39/24; A61M 39/26; A61M 2039/2473; A61M 2039/268

(57) ABSTRACT

The present invention is to a dripless fluid coupling device (a dripless connector) that can be used to driplessly connect and disconnect devices or assemblies to and from each other in a sterile manner. The dripless fluid coupling device of the present invention is easy to manufacture using known materials and methods; easy to use, requiring only a clamping device to connect and disconnect; sterilizable and relatively inexpensive compared to available prior art devices.

### CROSS-REFERENCE TO RELATED APPLICATIONS

The present application claims the benefit of priority of U.S. Provisional Patent Application No. 63/578,815, filed Aug. 25, 2023, the entire contents of which is incorporated herein by reference.

### BACKGROUND

The term dripless, or non-spill, describes a specific type of connector/disconnector designed to reduce spillage to zero or near zero when the couplings are disconnected. A number of industries count on dripless connectors to meet their requirements to protect equipment, patients, employees or the environment.

In the biotech industry many single use bioprocessing tubing and device assemblies are large and must be disassembled before disposal. What is needed are assemblies that can be quickly and driplessly disconnected after a production run allowing the single use device to be disposed of without complicated disassembly and unnecessary handling thereby saving time and money as well as providing a greater level of safety for the user.

Although dripless disconnector (also referred to as a dripless coupler in the art) devices are known in the art, they are not without limitations or are designed for specific uses. Many are designed for use over several to hundreds of runs. These are usually made of metal, are expensive and require cleaning and/or sterilization between uses. See, for example, U.S. Pat. No. 4,373,551 to Deere & Co.

Therefore, they are not suitable for use with single use devices. Others have been borrowed or adapted from the petroleum or chemical industries for use in the biotech industry. While serviceable, they are not designed with the needs of the biotech industry in mind, such as GMP level cleanliness, and may be needlessly complicated for use in the biotech industry.

Devices have also been made for specific use in the biotech industry. Examples are an irreversible pinch-pipe type crimp (e.g., NovaSeal™, manufactured by EMD Millipore Corporation, Burlington, MA), a reversible pinch clamp and/or thermal welding. Alternatively, other disconnection devices are available and known in the art such as QuickSeal® disconnect (Sartorius, Goettingen, Germany), Clipster® Aseptic Disconnector (Sartorius), AseptiQuik® DC (Colder Products Company, Arden Hills, MN), HFC39 (Colder Products Company), HFC Disconnect (Colder Products Company), Kleenpak™ Sterile Disconnector (PALL Corporation, Westborough, MA), SeriesLock™ (Eldon James, Denver, CO) and Lynx® CDR (EMD Millipore Corporation). However, these devices can be expensive, difficult to sanitize or use and/or have limited or specific functionality and may not be completely dripless.

What is needed in the biotech industry are quick connect/disconnect devices that are inexpensive, sterilizable, easy to make and use and dripless.

### SUMMARY OF THE INVENTION

The present invention solves these problems in the prior art. The dripless fluid coupling device of the present invention can, for example, come pre-assembled into a single use device assembly so that after a production run the device can be driplessly disconnected and disposed of. Further, the dripless fluid coupling device of the present invention is easy to manufacture using known materials and methods; easy to use, requiring only a clamping device to connect and disconnect; sterilizable and relatively inexpensive compared to available prior art devices.

In this regard, the present invention comprises, consists essentially of or consists of a dripless fluid coupling device, the dripless fluid coupling device comprising: a first coupler, said first coupler comprising a first fluid passage element having a linear direction, a first open end and a first coupling end; said first open end having a connector for attachment to a fluid source; said first coupling end ending in an angle relative to the linear direction of the first fluid passage element, said first fluid passage element having a fluid passage therethrough; a second coupler, said second coupler comprising a second fluid passage element having a linear direction, a second open end and a second coupling end; said second open end having a connector for attachment to a fluid source; said second coupling end ending in an angle relative to the linear direction of the second fluid passage element, said second fluid passage element having a fluid passage therethrough, the angle of the first coupling end of the first fluid passage element being complementary to the angle of the second coupling end of the second fluid passage element; wherein, when the first coupling end of the first fluid passage element and the second coupling end of the second fluid passage element are secured to each other they form a continuous fluid passage through the first fluid passage and the second fluid passage; the second coupler further comprising, a flow control passage, said flow control passage being at an angle relative to the linear direction of the second coupler, the flow control passage further comprising a flow control element, said flow control element being freely movable therein and wherein the flow control element is reversibly reconfigurable from an open configuration that allows passage of a fluid in in the continuous fluid passage to a closed configuration wherein said passage of the fluid in the continuous fluid passage is blocked.

The dripless fluid coupling device of the present invention may further comprise or consist essentially of a reversibly removable clamping device for securing the first coupler and the second coupler to each other.

The first fluid passage or the dripless fluid coupling device of the present invention may still further comprise or consist essentially of an extension extending out from the fluid passage that aligns with the flow control passage of the second coupler, wherein when the flow control element is in the closed position it extends into said flow control passage extension.

In another aspect, the flow control element of the dripless fluid coupling device has a first end and a second end, the first end of the flow control element being external to the flow control passage and the second end being internal to the flow control passage, the flow control element comprising a seal at the second end that can seal the fluid passage of the first coupler and the fluid passage of the second coupler when the flow control element when it is in the closed configuration.

In another aspect, the flow control element of the dripless fluid coupling device has a seal that is made from, for example, a polymer selected from one or more of silicone, rubber, including natural and synthetic rubbers, thermoplastic elastomers, polyolefins, polytetrafluorethylen (PTFE), thermoplastic perfluoropolymer resins, urethanes, ethylene propylene diene monomer (EPDM) rubber and polyvinylidene fluoride (PVDF) resins.

In another aspect, the connector for attachment to a fluid source of the dripless fluid coupling device of the present invention is selected from a group consisting of, for example, a barbed fitting, a ridged fitting, a friction fit, a Luer-lok® fitting, a threaded connection, a triclover connections and a Swagelok® fitting.

In another aspect of the dripless fluid coupling device of the present invention, the flow control element is a plunger.

In another aspect of the dripless fluid coupling device of the present invention, the flow control element is operated by one of direct pressure, mechanical pressure or gas pressure.

In another aspect of the dripless fluid coupling device of the present invention, the flow control element is controlled manually or automatically.

In another aspect of the dripless fluid coupling device of the present invention, the first fluid passage and said second fluid passage are selected from cylindrical, oblong, square or rectangular shapes when viewed in cross section.

In another aspect of the dripless fluid coupling device of the present invention, the seal comprises a first and a second segment, the first segment being positioned in the flow control passage of the second coupler and the second segment being positioned in the extension in the flow control passage extension of the first coupler when the flow control element is in the closed position.

In another aspect of the dripless fluid coupling device of the present invention, the first and the second couplers are made of one of more polymers.

In another aspect of the dripless fluid coupling device of the present invention, the polymer material is selected from one or more of recycled material(s) and/or recyclable material(s).

In another aspect of the dripless fluid coupling device of the present invention, the dripless fluid coupling device is a disposable, single use device.

In another embodiment, the present invention comprises, consists essentially of or consists of a first coupler, said first coupler comprising, consisting essentially of or consisting of a first fluid passage element having a linear direction, a first end and a second end; said first end having a connector for attachment to a fluid source; said second end ending in an angle relative to the linear direction of the first fluid passage element, said first fluid passage element having a fluid passage therethrough, said first coupler further comprising a first flow control passage housing a first flow control element (e.g., a plunger), wherein said first flow control element is freely movable therein and wherein said first flow control passage is at about 90 degrees to said first fluid passage; a second coupler, said second coupler comprising, consisting essentially of or consisting of a second fluid passage element having a linear direction, a first end and a second end; said first end having a connector for attachment to a fluid source; said second end ending in an angle relative to the linear direction of the second fluid passage element, said second fluid passage element having a fluid passage therethrough, said second coupler further comprising a second flow control passage housing a second flow control element (e.g., a plunger), wherein said second flow control element is freely movable therein and wherein said second flow control passage is about 90 degrees to said second fluid passage; wherein, the angle of the second end of the first fluid passage element is complementary to the angle of the second end of the second fluid passage element; wherein, when the second end of the first fluid passage element and the second end of the second fluid passage element are secured to each other a continuous fluid passage through the first fluid passage of the first coupler and the second fluid passage of the second coupler is formed; and wherein when the first and second flow control elements are depressed in said first and said second flow control passages, said flow of fluid in the fluid passage is blocked.

In another aspect of the dripless fluid coupling device of the present invention may further comprise or consist essentially of a reversibly removable clamping device for securing the first coupler and the second coupler to each other.

In another aspect of the dripless fluid coupling device of the present invention, the first and second flow control elements each have a first end and a second end, the first end of the flow control elements being external to the flow control passage and the second end being internal to the flow control passage, the first and second flow control elements comprising a seal at the second end that can seal the fluid passage of the first coupler and the fluid passage of the second coupler, respectively, when the plungers are in the closed configuration.

In another aspect of the dripless fluid coupling device of the present invention, the seal is made from a polymer selected from one or more of silicone, rubber, including natural and synthetic rubbers, thermoplastic elastomers, polyolefins, polytetrafluorethylen (PTFE), thermoplastic perfluoropolymer resins, urethanes, ethylene propylene diene monomer (EPDM) rubber and polyvinylidene fluoride (PVDF) resins.

In another aspect of the dripless fluid coupling device of the present invention, the connector for attachment to a fluid source is selected from a group consisting of a barbed fitting, a ridged fitting, a friction fit, a Luer-lok® fitting, a threaded connection, a triclover connections and a Swagelok® fitting.

In another aspect of the dripless fluid coupling device of the present invention, the flow control element(s) is(are) operated by one of direct pressure, mechanical pressure or gas pressure.

In another aspect of the dripless fluid coupling device of the present invention, the first flow control element and said second flow control element are controlled manually or automatically.

In another aspect of the dripless fluid coupling device of the present invention, the flow control element is a plunger.

In another aspect of the dripless fluid coupling device of the present invention, the first fluid passage and said second fluid passage are selected from cylindrical, oblong, square or rectangular shapes when viewed in cross section.

In another aspect of the dripless fluid coupling device of the present invention, the first and said second couplers are made of one of more polymers.

In another aspect of the dripless fluid coupling device of the present invention, the polymer material is selected from one or more of recycled material(s) and/or recyclable material(s).

In another aspect of the dripless fluid coupling device of the present invention, the first coupler and said second coupler are identical.

In another aspect of the dripless fluid coupling device of the present invention, the dripless fluid coupling device is a disposable, single use device.

In another embodiment, the present invention is toward a method of use of the fluid coupling device of the present invention, the method comprising, consisting essentially of or consisting of connecting two components of a bioprocessing train with a device of the present invention. In one embodiment, the devices are disposable single use devices. In another embodiment, the devices are suitable for at least one, more than one or for multiple uses. In other words, the devices may be used 1, 2, 3, 4, 5, 6, 7, 8, 9, 10, 15, 20, 30, 40, 50, 100 inclusively, or more times before being discarded. In this regard, the devices are sanitizable and/or sterilizable before the first use and/or between each use or after several uses.

In another aspect, the present invention is a method of fluidly connecting two devices, comprising: i) providing: a dripless fluid coupling device of the present invention; ii) connecting the first coupler of the dripless fluid coupling device to a first device and connecting the second coupler of the dripless fluid coupling device to a second device; and iii) connecting the first and second couplers of the dripless fluid coupling device to each other to produce a dripless connection.

In another aspect, the present invention is a method of fluidly connecting two devices, wherein connecting of the first and second couplers together is by clamping.

In another aspect, the present invention is a method of fluidly connecting two devices, wherein the dripless connection is sterile or sterilizable.

In another aspect, the present invention is a method of fluidly connecting two devices, wherein one of both of the two devices are single-use devices.

In another aspect, the present invention is a method of fluidly connecting two devices, wherein the devices are part of a bioprocessing train.

### BRIEF DESCRIPTION OF THE FIGURES

FIG. 1A shows one example of the device of the present invention. The plunger is retracted. FIG. 1B shows a cut-away view of the device of FIG. 1A.

FIG. 2A shows the device of FIG. 1 with the plunger depressed.

FIG. 2B shows a cut-away view of the device of FIG. 2A.

FIG. 3A shows the device of FIG. 1 with the clamp removed and the two parts (the first coupler and the second coupler) of the device separated. FIG. 3B shows a cut-away view of the device of FIG. 3A

FIGS. 4A and 4B shows another example of the device of the present invention wherein the plunger is spring activated. FIG. 4A shows a cut-away view with the plunger retracted. FIG. 4B shows a cut-away view with the plunger depressed.

FIGS. 5A and 5B shows another example of the device of the present invention wherein the plunger is activated with gas pressure.

FIG. 5A shows a cut-away view of the device where the plunger is retracted. FIG. 5B shows a cut-away view with the plunger depressed.

FIGS. 6A and 6B shows another example of the device of the present invention wherein two identical couplers are used. FIG. 6A shows an exploded view of one coupler showing the external shape and appearance, the plunger and the sealing element. FIG. 6B shows a transparent coupler with the plunger and sealing element retracted.

FIGS. 7A-7D shows the device of FIG. 6. FIG. 7A shows the identical first and second couplers separated from each other and with plungers retracted. FIG. 7Bshows the first and second couplers positioned together. A clamp is not represented in the figure for clarity.

FIG. 7C shows the coupled couplers with plungers depressed. FIG. 7D shows the now sealed and dripless couplers separated from each other.

### DETAILED DESCRIPTION OF THE INVENTION

When introducing elements of the present disclosure or the preferred embodiments(s) thereof, the articles “a,” “an,” “the” and “said” are intended to mean that there are one or more of the elements unless explicitly indicated otherwise. The terms “comprising,” “including” and “having” are intended to be inclusive and mean that there may be additional elements other than the listed elements.

The transitional phrases “comprising,” “consisting essentially of” and “consisting of” have the meanings as given in MPEP 2111.03 (Manual of Patent Examining Procedure; United States Patent and Trademark Office, 9^{th} ed., Feb. 2023 revision, R-07.2022). Any claims using the transitional phrase “consisting essentially of” will be understood as reciting only essential elements of the invention and any other elements recited in dependent claims are understood to be non-essential to the invention recited in the claim from which they depend.

The conjunctive expression “and/or” between multiple elements, as used herein, is understood to encompass both individual and combined options. For example, if Iwo elements are linked by “and/or,” a first option concerns the use of the first element without the second one. A second option concerns the use of the second element without the first one. A third option involves the use of the first and second elements together. It is understood that any one of these options falls within the meaning of the term and thus meets the conditions of the term “and/or” as used in this specification.

All numerical ranges include the recited values and all values within the cited range including all whole, fractional and decimal numbers, inclusive.

The terms “connector,” “disconnector” and “connector/disconnector” can be used interchangeably.

The device of the present invention may be referred to as the “fluid coupling device” or “dripless fluid coupling device” of the present invention. The fluid coupling device of the present invention is a dripless connector/disconnector suitable for the transport of fluids between vessels and/or devices and subsequent disconnection of the vessels and/or devices from each other in a dripless manner. The fluid coupling device of the present invention may also be capable of maintaining sterility of the related vessels and/or devices during fluid transport and subsequent disconnection of the vessels and/or devices from each other.

The present invention may be made from sustainable materials, recycled materials and/or recyclable materials, providing that such materials meet all requirements for usage of the device of the present invention in, for example, the research, biotech industry and/or pharmaceutical industry. Such requirements may be, for example, Good Manufacturing Practices (GMPs) as promulgated by the Food and Drug Administration (FDA). Further, the devices of the present invention may be made using renewable and/or sustainable energy sources, if available.

In one example of the present invention, a dripless disconnector is provided comprising a first coupler and a second coupler, each having a fluid passage having an open end and a coupling end. The first and second couplers are designed to mate or connect to each other. When the first and second couplers are connected to each other the fluid passages of the first and second couplers align and form a continuous fluid passage. The first and second couplers are held together by a clamp, clip or other suitable means known in the art. The ends of the couplers are angled relative to the fluid passage and complement each other in that the angle of one coupler matches the reverse or inverse angle of the angle of the other coupler (i.e., “complementary angles” or “angle(s) complementary to”). In other words, for example, if the angle of one coupler is 45 degrees relative to the axial fluid passage, the angle of the complementary coupler will be 135 degrees relative to the axial fluid passage. Although other complementary angles may be selected, the angles of 45 degrees and 135 degrees are preferred. The mating surfaces of the coupling ends are machined so that they form a leak-free seal when mated and coupled or connected (e.g., clamped) to each other. In some instances, a resilient member (e.g., a seal) may be used/positioned between the two couplers. In some other instances, the surface of the angled ends of one or both of the couplers may have a groove for retaining a resilient member (e.g., an O-ring, washer or other type of seal known to one of skill in the art) to better provide a leak proof seal at the interface when the first and second couplers are connected. If both couplers have a groove in the surface of the angled ends, the grooves are positioned so that they align with each other when the couplers are coupled to each other. In this way, the resilient member will be positioned in the groove in each of the respective couplers. The resilient members or seals used in the embodiments disclosed herein can be made of, but not limited to, silicone, rubber, including natural and synthetic rubbers, thermoplastic elastomers, polyolefins, polytetrafluorethylen (PTFE), thermoplastic perfluoropolymer resins, urethanes, ethylene propylene diene monomer (EPDM) rubber, polyvinylidene fluoride (PVDF) resins, etc., as known to one of skill in the art.

The ends of the first and second couplers opposite the angled ends (the first and second open ends, respectively) have a means to attach to a fluid source. For example, a barbed fitting, one or more ridges circumventing the one end to provide a gripping surface for tubing (a “ridged” fitting), a straight tube wherein tubing can be retained by a friction fit (a “friction fit” or “interference fit” fitting) and other press fit, interference fit or friction fit connections, a Luer-lok® fitting, a tapered fitting, a threaded connection, a triclover connection, a Swagelok® fitting and a twist connect/disconnect connection, as are known to one of skill in the art. Suitable configurations can include a combination of connection types with the first and second couplers each having a different type of attachment means or, in another example, both the first and second couplers have the same means of attachment to a fluid source. For the purposes of this invention, the term “fluid source” refers to both fluids flowing into the dripless coupling device of the present invention and/or out of the dripless coupling device of the present invention.

The dripless disconnector of the present invention also comprises a flow control passage to allow the flow of fluid through the continuous fluid passage to be sealed or stopped at the juncture of the first and second coupler when, for example, the first and second couplers are disconnected after use, for example, after having fluid pass through the continuous fluid passage. The flow control passage is located in one of the flow control couplers at an angle relative to the flow passage. In other words, the flow control passage is at an angle relative to the linear direction of the continuous fluid passage of the coupler.

The dripless disconnect of the present invention is connectable and disconnectable (i.e., the first and second couplers can be coupled/connected to each other and are disconnectable or can be separated from each other). When the flow control element, e.g., a plunger, is in the closed position, the fluid passage of the dripless disconnector of the present invention is sealed. Both the first coupler and the second coupler may be sealed preventing any fluid in the first coupler and second coupler from spilling or leaking from the first coupler and second coupler (i.e., dripless), respectively. As an alternative, if only the first sealing element is present, the flow passage of the first coupler is sealed and the flow path of the second coupler, while not sealed, may be drained completely prior to being disconnected. In some instances, the coupler is 100% dripless with no loss of fluid. In other instances, there may be a negligeable amount (e.g., about 0.01-about 0.05 ml) of residual fluid between the couplings (i.e., between the seals), and thus, once disconnected, there may be slight spillage of this residual fluid. For the present invention, this is considered to be dripless.

In one example, the flow control element is located in the flow control passage and is a plunger. The plunger may comprise a first end and a second end. The first end may comprise a handle, tab or other means for ease in moving the plunger within the flow control passage. The second end may comprise a resilient member for ensuring a tight seal to prevent leaks when the plunger is both in the open position (preventing fluid from leaking out of the flow control passage) or closed position (preventing fluid from leaking out of the device's open end (if not connected to a fluid source), coupling end and flow control passages). In an alternative version, the plunger and flow control element have a machine fit (i.e., are machined to have a clearance between the plunger and inner wall of the flow control element that is close enough to prevent leaking). One of skill in the art can determine the exact tolerance for a leak-proof machine fit for fluids such as those used in the biotech industry (e.g., aqueous fluids such as cell culture media, saline, sanitization fluids, etc.).

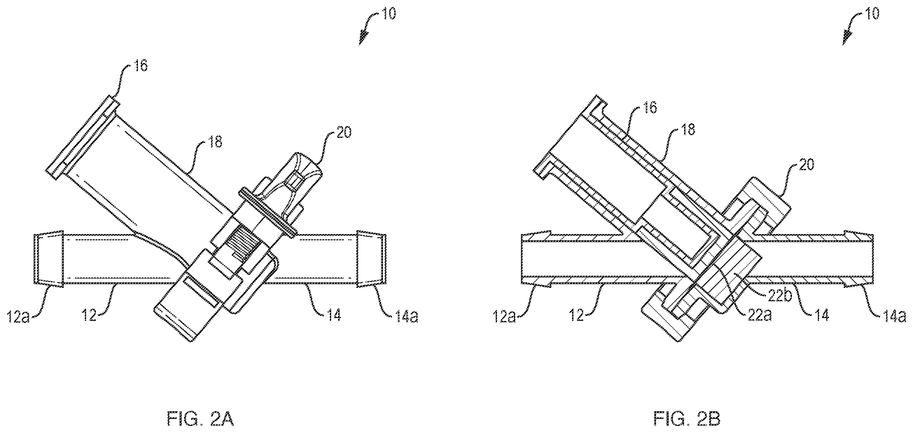

In other instances, the flow control element may further comprise a second resilient member at the second end of the flow control element. The second resilient member is in contact with the first resilient member when the flow control element is in the open configuration. The second resilient member is not attached or affixed to the first resilient member (or, in some instances, may be lightly attached such as with a tacky biocompatible substance, as is known in the art, or static interaction). When in the closed configuration, the second resilient member is pushed into a recess located at the angled end of the first coupler (see, for example, FIG. 2B) and, when the first and second couplers are disconnected, remains in the recess sealing the fluid flow path of the first coupler. The resilient members of the flow control element are held in place by a slight interference fit (i.e., by a friction fit) between the resilient member and the inner walls of the device. The resilient members (i.e., seals) used in the embodiments disclosed herein can be made of, but not limited to, silicone, rubber, including natural and synthetic rubbers, thermoplastic elastomers, polyolefins, PTFE, thermoplastic perfluoropolymer resins, urethanes, EPDM rubber, PDDF resins, etc., as known to one of skill in the art.

In another example of the dripless disconnector of the present invention, the first and second resilient members may be pushed into the closed position by means other than a manually operated flow control element (for example, a plunger). In one example, the first and second resilient members are pushed into the closed position by means of a spring. The spring may be in a compressed state and only allowed to expand when the compression is released by a user. For example, the spring may be released when a knob is turned (e.g., a quarter of a full turn) releasing or moving a tab or tang keeping the spring compressed and thereby allowing the spring to expand.

In another example of the dripless disconnect of the present invention, the resilient members may be pushed into the closed position by expanding gas. Compressed gas may be retained in a small or miniaturized cylinder and released by a user when desired. Examples of miniaturized compressed gas cylinders known to one of skill in the art and suitable for use with the present invention include (but are not limited to) those made by PICOCYL (Golden, CO), Leland (Plainfield, NJ) and iSi Components GmbH (Vienna, Austria).

In yet another example of the present invention, the flow control element may be controlled manually or may be controlled by an automated control system.

The dripless disconnect of the present invention may be made of any suitable material known to one of skill in the art. Plastic polymers are preferred as they are readily available, inexpensive, sterilizable and recyclable. Examples of suitable polymers include (but are not limited to) materials capable of withstanding the conditions typically encountered by such devices, including those of sterilization. Suitable materials include, but are not limited to, plastics and metals (including, but not limited to, stainless steel, aluminum and brass).

Suitable plastic materials may include, but are not limited to, polysulfone, glass filled polysulfone, polyphenylene sulfide, glass filled polyphenylene sulfide, polyphenyl sulfone and glass filled polyphenyl sulfone. These are all acceptable materials due to their biocompatibility as well as their chemical, heat and creep resistance. The components of the device of the present invention may be formed by machining or molding, or a combination thereof, as is known to one of skill in the art.

Examples of the present invention are illustrated in the figures. The figures and descriptions thereof do not limit the present invention but serve only to illustrate certain embodiments of the present invention. One of skill in the art, in view of and with the teachings of this specification, would understand that variations of the invention as shown in the figures and as described herein are possible without diverging from the scope or breadth of the invention in view of the teachings of this specification.

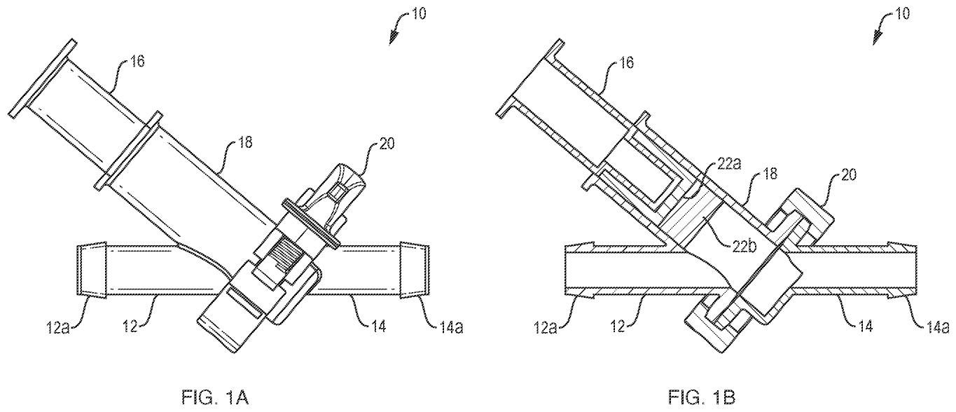

FIG. 1A shows one example of the present invention 10. In this external view, the first coupler 14 is attached to the second coupler 18 by a clamp 20. The first end of the flow control element 16, here represented by a plunger, can be seen extending from the second coupler 18 in the open position. The tube 14 housing the fluid flow path of the first coupler and the tube 12 housing the fluid flow path of the second coupler are shown, each having a means (12a and 14a; here, a barbed fitting) for connecting to a fluid source.

FIG. 1B shows a cross sectional view of the device 10 shown in FIG. 1A. Shown in this view are the first resilient piece 22a and second resilient piece 22b. As mentioned, above, the first and second resilient pieces are held by an interference fit. The first and second resilient pieces can be made from natural or synthetic rubber or plastics, as detailed above. One of skill in the art will be able to determine the degree of resilience needed to provide both a suitable interference fit and a dripless seal when the plunger is in the open position and when the plunger is depressed and in the closed position (as shown in FIGS. 2A and 2B. The interference fit is, in one embodiment, similar to that found in a standard syringe.

FIG. 2A shows the device 10 of FIGS. 1A and 1B when the plunger is in the closed position. Numbering is the same as in FIGS. 1A and 1B. FIG. 2B is a cross sectional view of the device with the plunger in the closed position. The positions of the first resilient piece 22a and second resilient piece 22b when the plunger is in the closed position are shown.

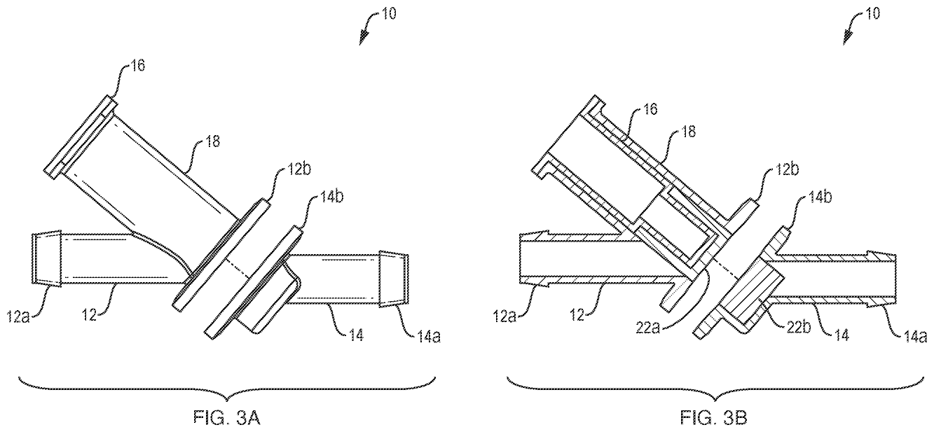

FIGS. 3A and 3B show the device 10 of FIGS. 1A and 1B when the plunger is in the closed position and the first coupler and second coupler are disconnected from each other. The numbering is the same as in FIGS. 1A and 1B. As shown in the figure, the second resilient piece 22b stays in the second coupler with a friction or interference fit. Likewise, the first resilient piece 22a stays in the first coupler. In this way, both the first and second couplers are sealed and any fluid therein is contained and prevented from leaking or dripping out of the disconnected couplers.

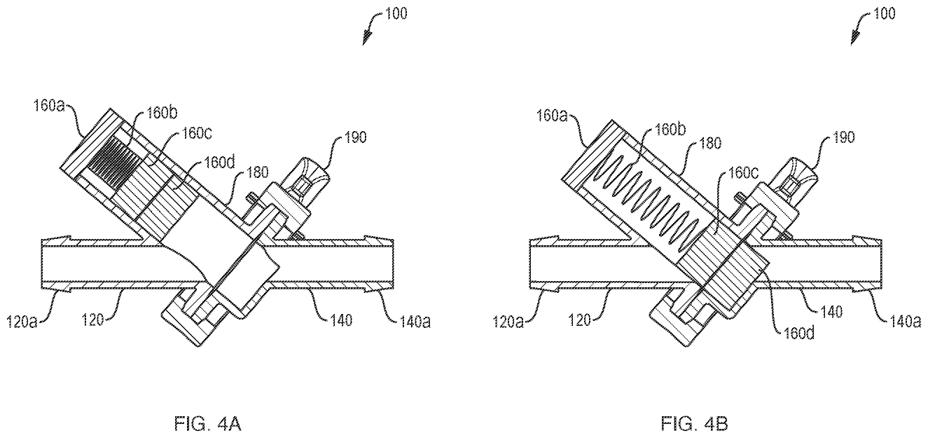

In another aspect of the invention, the plunger is operated by pressure generated by releasing the tension in a compressed spring. FIGS. 4A and 4B show this aspect of the invention. Both figures show a cross sectional view of the device 100. In FIG. 4A the plunger 160a is in the open position. The spring 160b in the compressed state, the first resilient piece 160c and the second resilient piece 160d are shown. In FIG. 4B the spring 160b is in the relaxed state and the plunger is in the closed position. The first resilient 160c and second resilient 160d pieces are shown in their respective locations in the first 120 and second 140 couplers for sealing the device when the first and second couplers are disconnected. It can be seen that the interface between the first and second resilient pieces align with the interface between the first and second couplers. As with the device of the present invention with the manually operated plunger shown in FIGS. 1A-3B , the first and second resilient pieces are retained in their respective couplers by a friction or interference fit.

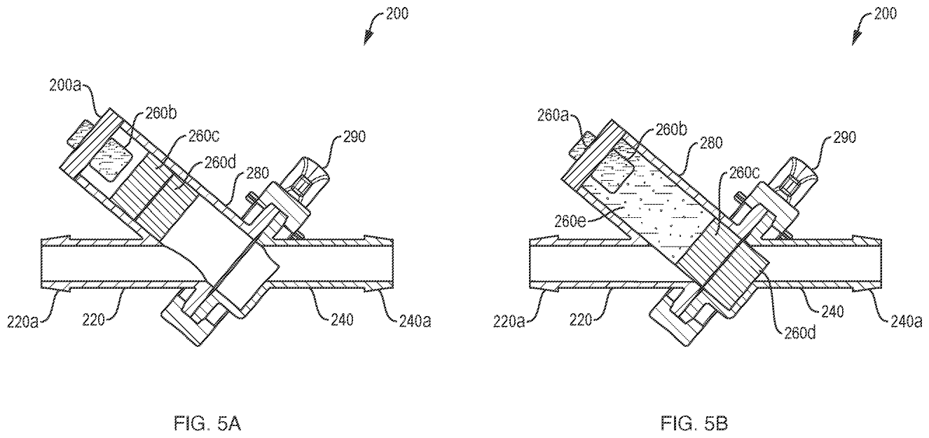

In another aspect of the present invention, the plunger is operated by gas pressure. FIGS. 5A and 5B show this aspect of the invention. Both figures show a cross sectional view of the device. In FIG. 5A the plunger 260a is in the open position. The compressed gas cylinder 260b, the first resilient piece 260c and the second resilient piece 260d are shown. In FIG. 5B the gas cylinder 160b has been activated (i.e., the compressed gas released) forcing the plunger into the closed position. The first resilient 260c and second resilient 260d pieces are shown in their respective locations in the first 220 and second 240 couplers for sealing the device 200 when the first and second couplers are disconnected. It can be seen that the interface between the first and second resilient pieces align with the interface between the first and second couplers. As with the device of the present invention with the manually operated plunger shown in FIGS. 1A-3B, the first and second resilient pieces are retained in their respective couplers by a friction or interference fit.

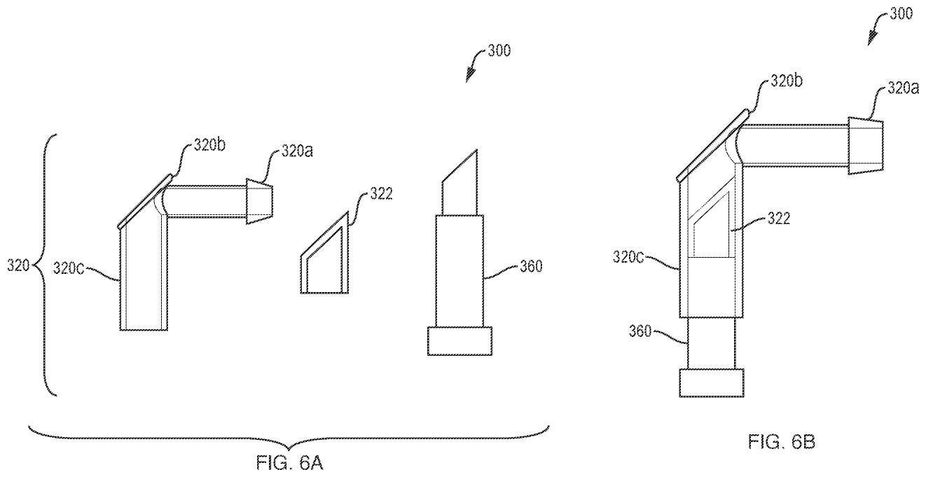

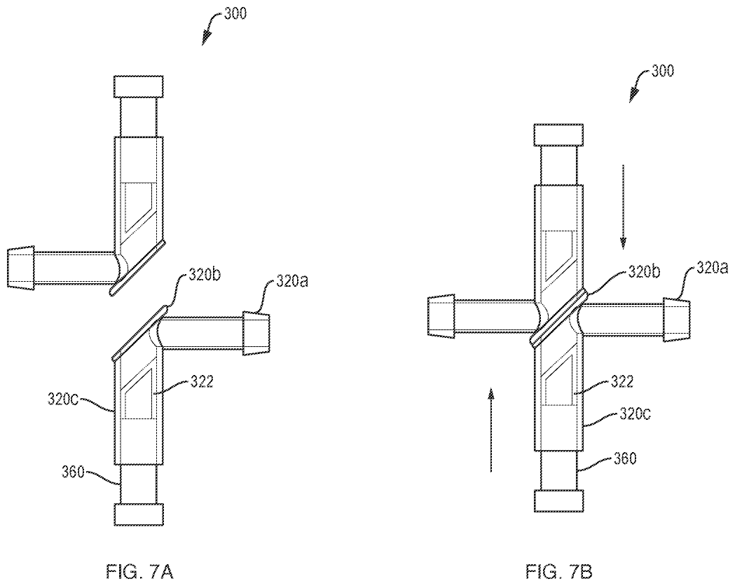

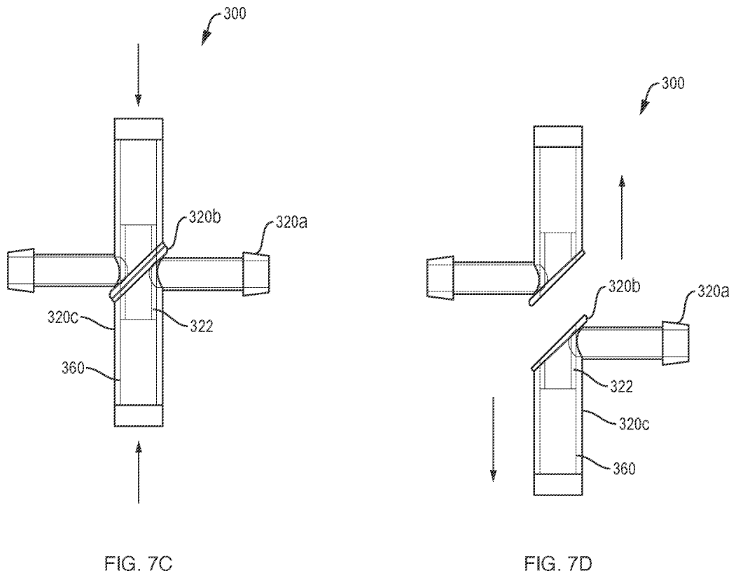

FIGS. 6 and 7 show yet another aspect of the present invention. Unlike the devices represented by the illustrations of FIGS. 1A-5B, the device as illustrated in FIGS. 6A & B and 7A-D utilizes a first and a second coupler that are identical or substantially identical in design. FIG. 6A shows a coupler 300 of this aspect of the invention. The coupler in this view is disassembled. The housing 320 has an end for connecting to a fluid source 320a and an angled end 320b located at the angle between the end for connecting to a fluid source 320a and 17 the end that houses the plunger 320c. The plunger 360 and resilient piece 322 are also. FIG. 6B shows the coupler of this device assembled with the resilient piece 322 positioned on the plunger 360 and the assembled plunger and resilient piece located in the coupler 320 in the open position shown (as if the housing shown is transparent).

For FIGS. 6B & 7A-7D , the internal parts are shown as though the coupler is transparent for ease of visualization. FIG. 7A shows two of the couplers of this aspect of the invention separated from each other but aligned for attachment to each other. The numbering is as in FIGS. 6A & 6B. FIG. 7B shows the two couplers brought into contact with each other. A clamp, clip or other reversible joining means (not shown) holds the two couplers together, similarly to the device of FIG. 1A. Fluid passes through the device in an axial direction, i.e., left to right or right to left in the figure.

FIG. 7C illustrates the operation of the device with the plungers 360 of both couplers moved to the closed position thereby causing the resilient pieces 322 to move to the angled end 320b sealing the respective couplers. FIG. 7D shows the separation of the couplers (disconnected) with the respective plungers 360 of the devices 300 in the closed position and with the respective resilient pieces 320b sealing the couplers. It can be appreciated that only one of the plungers may be activated, or only one coupler comprises a plunger. In this scenario, the second coupler is drained prior to the couplers being disconnected.

What is claimed is:

1. A dripless fluid coupling device, comprising:
a first coupler, said first coupler comprising a first fluid passage element having a linear direction, a first open end and a first coupling end; said first open end having a connector for attachment to a fluid source; said first coupling end ending in an angle relative to the linear direction of the first fluid passage element, said first fluid passage element having a fluid passage therethrough;
a second coupler, said second coupler comprising a second fluid passage element having a linear direction, a second open end and a second coupling end; said second open end having a connector for attachment to a fluid source; said second coupling end ending in an angle relative to the linear direction of the second fluid passage element, said second fluid passage element having a fluid passage therethrough, the angle of the first coupling end of the first fluid passage element being complementary to the angle of the second coupling end of the second fluid passage element;
wherein, when the first coupling end of the first fluid passage element and the second coupling end of the second fluid passage element are secured to each other they form a continuous fluid passage through the first fluid passage and the second fluid passage;
the second coupler further comprising, a flow control passage, said flow control passage being at an angle relative to the linear direction of the second coupler, the flow control passage further comprising a flow control element, said flow control element being freely movable therein and wherein the flow control element is reversibly reconfigurable from an open configuration that allows passage of a fluid in in the continuous fluid passage to a closed configuration wherein said passage of the fluid in the continuous fluid passage is blocked.

2. The dripless fluid coupling device of claim 1, further comprising; a reversibly removable clamping device for securing the first coupler and the second coupler to each other.

3. The dripless fluid coupling device of claim 1, wherein the first fluid passage has an extension extending out from the fluid passage that aligns with the flow control passage of the second coupler, wherein when the flow control element is in the closed position it extends into said flow control passage extension.

4. The dripless fluid coupling device of claim 3, wherein the flow control element has a first end and a second end, the first end of the flow control element being external to the flow control passage and the second end being internal to the flow control passage, the flow control element comprising a seal at the second end that can seal the fluid passage of the first coupler and the fluid passage of the second coupler when the flow control element when it is in the closed configuration.

5. The dripless fluid coupling device of claim 4, wherein said seal is made from a polymer selected from one or more of silicone, rubber, including natural and synthetic rubbers, thermoplastic elastomers, polyolefins, polytetrafluorethylen (PTFE), thermoplastic perfluoropolymer resins, urethanes, ethylene propylene diene monomer (EPDM) rubber and polyvinylidene fluoride (PVDF) resins.

6. The dripless fluid coupling device of claim 1, wherein said connector for attachment to a fluid source is selected from a group consisting of a barbed fitting, a ridged fitting, a friction fit, a Luer-lok® fitting, a threaded connection, a triclover connection and a Swagelok® fitting.

7. The dripless fluid coupling device of claim 1, wherein said flow control element is a plunger.

8. The dripless fluid coupling device of claim 1, wherein said flow control element is operated by one of direct pressure, mechanical pressure or gas pressure.

9. The dripless fluid coupling device of claim 1, wherein said flow control element is controlled manually or automatically.

10. The dripless fluid coupling device of claim 1, wherein said first fluid passage and said second fluid passage are selected from cylindrical, oblong, square or rectangular shapes when viewed in cross section.

11. The dripless fluid coupling device of claim 4, wherein said seal comprises a first and a second segments, the first segment being positioned in the flow control passage of the second coupler and the second segment being positioned in the extension in the flow control passage extension of the first coupler when the flow control element is in the closed position.

12. The dripless fluid coupling device of claim 1, wherein said first and said second couplers are made of one of more polymers.

13. The dripless fluid coupling device of claim 12, wherein said polymer material is selected from one or more of recycled material and/or recyclable material.

14. The dripless fluid coupling device of claim 1, wherein said dripless fluid coupling device is a disposable, single use device.

15. A dripless fluid coupling device, comprising:
a first coupler, said first coupler comprising a first fluid passage element having a linear direction, a first end and a second end; said first end having a connector for attachment to a fluid source; said second end ending in an angle relative to the linear direction of the first fluid passage element, said first fluid passage element having a fluid passage therethrough, said first coupler further comprising a first flow control passage housing a first flow control element wherein said first flow control element is freely movable therein and wherein said first flow control passage is at about 90 degrees to said first fluid passage;
a second coupler, said second coupler comprising a second fluid passage element having a linear direction, a first end and a second end; said first end having a connector for attachment to a fluid source; said second end ending in an angle relative to the linear direction of the second fluid passage element, said second fluid passage element having a fluid passage therethrough, said second coupler further comprising a second flow control passage housing a second flow control element, wherein said second flow control element is freely movable therein and wherein said second flow control passage is about 90 degrees to said second fluid passage;
wherein, the angle of the second end of the first fluid passage element is complementary to the angle of the second end of the second fluid passage element; wherein, when the second end of the first fluid passage element and the second end of the second fluid passage element are secured to each other a continuous fluid passage through the first fluid passage of the first coupler and the second fluid passage of the second coupler is formed;
and wherein when the first and second flow control elements are depressed in said first and said second flow control passages, said flow of fluid in the fluid passage is blocked.

16. The dripless fluid coupling device of claim 15, further comprising; a reversibly removable clamping device for securing the first coupler and the second coupler to each other.

17. The dripless fluid coupling device of claim 16, wherein the first and second flow control elements each have a first end and a second end, the first end of the flow control elements being external to the flow control passage and the second end being internal to the flow control passage, the first and second flow control elements comprising a seal at the second end that can seal the fluid passage of the first coupler and the fluid passage of the second coupler, respectively, when the flow control elements are in the closed configuration.

18. The dripless fluid coupling device of claim 15, wherein said seal is made from a polymer selected from one or more of silicone, rubber, including natural and synthetic rubbers, thermoplastic elastomers, polyolefins, polytetrafluorethylen (PTFE), thermoplastic perfluoropolymer resins, urethanes, ethylene propylene diene monomer (EPDM) rubber and polyvinylidene fluoride (PVDF) resins.

19. The dripless fluid coupling device of claim 15, wherein said connector for attachment to a fluid source is selected from a group consisting of a barbed fitting, a ridged fitting, a friction fit, a Luer-lok® fitting, a threaded connection, a triclover connection and a Swagelok® fitting.

20. The dripless fluid coupling device of claim 15, wherein said flow control elements are operated by one of direct pressure, mechanical pressure or gas pressure.

21. The dripless fluid coupling device of claim 20, wherein said first flow control element and said second flow control element are controlled manually or automatically.

22. The dripless fluid coupling device of claim 15, wherein said flow control element is a plunger.

23. The dripless fluid coupling device of claim 15, wherein said first fluid passage and said second fluid passage are selected from cylindrical, oblong, square or rectangular shapes when viewed in cross section.

24. The dripless fluid coupling device of claim 15, wherein said first and said second couplers are made of one of more polymers.

25. The dripless fluid coupling device of claim 24, wherein said polymer material is selected from one or more of recycled material and/or recyclable material.

26. The dripless fluid coupling device of claim 15, wherein said first coupler and said second coupler are identical.

27. The dripless fluid coupling device of claim 12, wherein said dripless fluid coupling device is a disposable, single use, device.

28. A method of fluidly connecting two devices, comprising: i) providing:
the dripless fluid coupling device of claim 1; ii) connecting the first coupler of the dripless fluid coupling device to a first device and connecting the second coupler of the dripless fluid coupling device to a second device; and iii) connecting the first and second couplers of the dripless fluid coupling device to each other to produce a dripless connection.

29. The method of claim 28, wherein said connection of the first and second couplers is by clamping.

30. The method of claim 28, wherein said dripless connection is sterile or sterilizable.

31. The method of claim 28, wherein one of both of the two devices are single-use devices.

32. The method of claim 28, wherein said devices are part of a bioprocessing train.

33. The method of claim 28, wherein step iii) is performed before step ii).

34. A method of fluidly connecting two devices, comprising: i) providing: the dripless fluid coupling device of claim 15; ii) connecting the first coupler of dripless fluid coupling device to a first device and connecting the second coupler of the dripless fluid coupling device to a second device; and iii) clamping the first and second couplers of the dripless fluid coupling device to each other to produce a dripless connection.

35. The method of claim 34, wherein said dripless connection is sterile or sterilizable.

36. The method of claim 34, wherein one or both of the two devices are single-use devices.

37. The method of claim 34, wherein said devices are part of a bioprocessing train.

38. The method of claim 34, wherein said connection of the first and second couplers is by clamping.

39. The method of claim 34, wherein step iii) is performed before step ii).
